(19) United States
(12) Patent Application Publication
(10) Pub. No.: US 2025/0029243 A1
(43) Pub. Date: Jan. 23, 2025

(54) ULTRASOUND IMAGE PROCESSING APPARATUS

(71) Applicant: FUJIFILM Healthcare Corporation, Kashiwa-shi (JP)

(72) Inventors: Jun Yasuda, Tokyo (JP); Tomohiko Tanaka, Tokyo (JP); Tetsuya Yamada, Chiba (JP)

(21) Appl. No.: 18/758,776

(22) Filed: Jun. 28, 2024

(30) Foreign Application Priority Data
Jul. 21, 2023 (JP) 2023-119345

(51) Int. Cl. G06T 7/00; A61B 8/08

(52) U.S. Cl. CPC G06T 7/0012; A61B 8/488; G06T 2207/10132; G06T 2207/20081

(57) ABSTRACT

A Doppler signal processing unit acquires target power distribution information indicating a power distribution of a Doppler signal. A texture parameter specifying unit inputs the target power distribution information to a learning model. The trained learning model outputs a texture parameter appropriate for the target power distribution information. The texture parameter is a parameter indicating a feature of a gamma curve. An appropriate gamma curve decision unit decides an appropriate gamma curve that is appropriate for the target power distribution information, based on the texture parameter appropriate for the target power distribution information. An image formation unit forms a power Doppler image based on the target power distribution information and the appropriate gamma curve.

### CROSS REFERENCE TO RELATED APPLICATION

This application claims priority to Japanese Patent Application No. 2023-119345 filed on Jul. 21, 2023 which is incorporated herein by reference in its entirety including the specification, claims, abstract, and drawings.

### BACKGROUND OF THE INVENTION

### 1. Field of the Invention

The present specification discloses an improvement of an ultrasound image processing apparatus.

### 2. Description of the Related Art

In the related art, there is known an ultrasound diagnostic apparatus that transmits and receives ultrasound waves to and from a subject, forms an ultrasound tomographic image based on a reception signal obtained by the transmission and reception of the ultrasound waves, and displays the formed ultrasound tomographic image (that is, a B-mode image) on a display. In such an ultrasound diagnostic apparatus, a Doppler signal is formed from a reception signal by using a Doppler effect in a received wave from a subject, and a Doppler image is formed based on the Doppler signal. The Doppler image includes a color Doppler image in which a velocity and a direction of a blood flow in the subject are shown by color, and a power Doppler image in which the power of the Doppler signal is shown by brightness or color. In general, the Doppler image is displayed in a superimposed form on the ultrasound tomographic image.

Here, the Doppler signal may include a signal component indicating a movement of a subject tissue (for example, a cardiac wall) in addition to a blood flow signal indicating the blood flow. Such a signal component will be referred to as a clutter signal. In general, the clutter signal is a noise component that lowers the visibility of the blood flow signal in the Doppler image. Therefore, in the related art, a technology of suppressing the clutter signal has been proposed.

For example, JP2023-003852A discloses an ultrasound imaging apparatus that discriminates between a clutter signal and a blood flow signal in a reception signal of a ultrasound wave based on a feature value, which is a parameter (for example, an amplitude in a packet direction of an IQ signal) indicating a physical difference between the clutter signal and the blood flow signal, generates a suppression map for suppressing the clutter signal based on the discrimination result, and applies the suppression map to the reception signal to suppress the clutter signal.

### SUMMARY OF THE INVENTION

As described above, the Doppler image includes an image (power Doppler image) in which the power of the Doppler signal is shown by the color or the brightness, or an image (color Doppler image) in which the blood flow or the velocity of the tissue of the subject indicated by the Doppler signal is shown by the color. Therefore, in a case of forming the Doppler image, the power of the Doppler signal or the velocity indicated by the Doppler signal is transformed into a pixel value by using a gamma curve indicating a relationship between the power of the Doppler signal or the velocity indicated by the Doppler signal, and the pixel value in the Doppler image.

FIG. 8 is a diagram showing a first example of the gamma curve. In FIG. 8, a horizontal axis represents a power value of the Doppler signal, and a vertical axis represents a frequency or the pixel value. A graph of a one-dot chain line indicates a power value and a frequency of the clutter signal included in the Doppler signal, and a graph of a solid line indicates a power value and a frequency of the blood flow signal included in the Doppler signal. In general, since the power value of the clutter signal is small and the power value of the blood flow signal is large, the clutter signals and the blood flow signals are distributed as shown in FIG. 8.

A graph of a thick solid line in FIG. 8 is a gamma curve showing a relationship between the power value and the pixel value. According to the gamma curve as shown in FIG. 8, the power value smaller than a clutter suppression cutoff value is transformed into a minimum pixel value PVₘᵢₙ regardless of the power value, and the power value larger than a blood flow signal cutoff value is transformed into a maximum pixel value PVₘₐₓ regardless of the power value. That is, a difference in the power value in a power region smaller than the clutter suppression cutoff value is not expressed by the pixel value, and a difference in the power value in a power region larger than the blood flow signal cutoff value is not expressed by the pixel value.

On the other hand, according to the gamma curve of FIG. 8, the power value included in a dynamic range between the clutter suppression cutoff value and the blood flow signal cutoff value is transformed into the pixel value corresponding to the power value. That is, as for the power value between the clutter suppression cutoff value and the blood flow signal cutoff value, the difference in the power value is expressed as the pixel value. This case is also referred to as the power value being expressed in gradations. It should be noted that, in the example of FIG. 8, the gamma curve in the dynamic range is a straight line connecting a point A (power value, pixel value)=(clutter suppression cutoff value, PVₘᵢₙ) and a point B (power value, pixel value)=(blood flow signal cutoff value, PVₘₐₓ), but may be a curve connecting the point A and the point B.

The gamma curve can be set by a user. In particular, the clutter suppression cutoff value and the signal suppression cutoff value can be set by the user. Here, there is a problem that an appropriate Doppler image (in this case, the power Doppler image) cannot be obtained in a case in which an appropriate gamma curve is not set according to the Doppler signal (more specifically, the power distributions of the clutter signal and the blood flow signal).

For example, as shown in FIG. 8, in a case in which the gamma curve is set such that a major part of the blood flow signal is larger than the blood flow signal cutoff value, the major part of the blood flow signal is expressed by the maximum pixel value PVₘₐₓ, and thus the power value of the blood flow signal cannot be successfully expressed in gradations in the power Doppler image. In addition, as shown in FIG. 9, in a case in which the clutter suppression cutoff value is set such that many clutter signals are included in the dynamic range, the clutter signal is significantly present in the power Doppler image, and the visibility of the blood flow signal is lowered.

It should be noted that, in FIGS. 8 and 9, the horizontal axis represents the power value, and the gamma curve in a case in which the power Doppler image is formed as the Doppler image is shown. However, even in a case in which, in FIGS. 8 and 9, the horizontal axis represents the velocity (in other words, the Doppler frequency) indicated by the Doppler signal, and the color Doppler image is formed as the Doppler image, the same problem as described above may occur.

An object of the ultrasound image processing apparatus disclosed in the present specification is to automatically set an appropriate gamma curve indicating a relationship between power of a Doppler signal or a velocity indicated by the Doppler signal, and a pixel value in a Doppler image.

The present specification discloses an ultrasound image processing apparatus comprising: a texture parameter specifying unit that inputs target power distribution information to be processed to a learning model trained using, as training data, a combination including power distribution information indicating a power distribution of a Doppler signal obtained by transmitting and receiving an ultrasound wave to and from a subject, and a texture parameter indicating a gamma curve, which indicates a relationship between power of the Doppler signal and a pixel value in a Doppler image and is appropriate for the power distribution information, to predict the texture parameter appropriate for the power distribution information and output the predicted texture parameter in a case in which the power distribution information is input, and that specifies the texture parameter appropriate for the target power distribution information; an appropriate gamma curve decision unit that decides an appropriate gamma curve that is the gamma curve appropriate for the target power distribution information, based on the specified texture parameter; and a Doppler image formation unit that forms a Doppler image in which the power of the Doppler signal is shown, based on the target power distribution information and the appropriate gamma curve.

The ultrasound image processing apparatus may further comprise: a training processing unit that uses, as the training data, a combination including the power distribution information and the texture parameter indicating the gamma curve set by a user, to train the learning model to predict the texture parameter appropriate for the user and output the predicted texture parameter in a case in which the power distribution information is input, in which the texture parameter specifying unit inputs the target power distribution information to the learning model, to specify the texture parameter appropriate for the user, and the appropriate gamma curve decision unit decides the appropriate gamma curve that is appropriate for the user, based on the specified texture parameter.

The training processing unit may present a plurality of the combinations including the power distribution information and the texture parameter, which are stored in advance in a memory, to the user, and use, as the training data, a combination including the power distribution information and the texture parameter, which is selected by the user, to train the learning model.

The appropriate gamma curve decision unit may select the appropriate gamma curve based on the gamma curve calculated based on the texture parameter specified by the texture parameter specifying unit from among a plurality of types of the gamma curves stored in advance in a memory.

The appropriate gamma curve decision unit may notify a user of the decided appropriate gamma curve.

The present specification also discloses an ultrasound image processing apparatus comprising: a texture parameter specifying unit that inputs target velocity distribution information to be processed to a learning model trained using, as training data, a combination including velocity distribution information indicating a velocity distribution indicated by a Doppler signal obtained by transmitting and receiving an ultrasound wave to and from a subject, and a texture parameter indicating a gamma curve, which indicates a relationship between a velocity indicated by the Doppler signal and a pixel value in a Doppler image, to predict the texture parameter appropriate for the velocity distribution information and output the predicted texture parameter in a case in which the velocity distribution information is input, and that specifies the texture parameter appropriate for the target velocity distribution information; an appropriate gamma curve decision unit that decides an appropriate gamma curve that is the gamma curve appropriate for the target velocity distribution information, based on the specified texture parameter; and a Doppler image formation unit that forms a Doppler image in which a velocity of a blood flow or a tissue of the subject is shown, based on the target velocity distribution information and the appropriate gamma curve.

With the ultrasound image processing apparatus disclosed in the present specification, it is possible to automatically set the appropriate gamma curve indicating the relationship between the power of the Doppler signal or the velocity indicated by the Doppler signal, and the pixel value in the Doppler image.

### BRIEF DESCRIPTION OF THE DRAWINGS

FIG. 1 is a schematic configuration diagram of an ultrasound diagnostic apparatus according to the present embodiment.

FIG. 2 is a diagram showing power distribution information and a gamma curve.

FIG. 3 is a conceptual diagram showing processing of training a learning model.

FIG. 4 is a flowchart showing a flow of the processing of training the learning model.

FIG. 5 is a diagram showing an example of a selection screen of training data.

FIG. 6 is a conceptual diagram showing processing of the learning model.

FIG. 7 is a flowchart showing a flow of a method of deciding an appropriate gamma curve.

FIG. 8 is a second diagram showing an example of the gamma curve.

FIG. 9 is a third diagram showing an example of the gamma curve.

### DESCRIPTION OF THE PREFERRED EMBODIMENTS

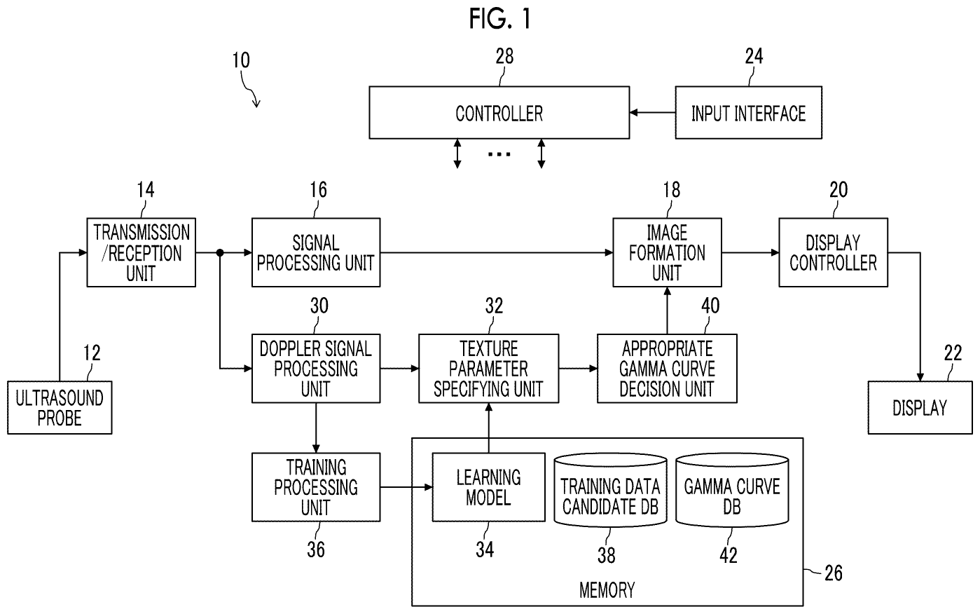

FIG. 1 is a schematic configuration diagram of an ultrasound diagnostic apparatus 10 as an ultrasound image processing apparatus according to the present embodiment. The ultrasound diagnostic apparatus 10 is a medical apparatus that is installed in a medical institution, such as a hospital, and is used during an ultrasound examination.

The ultrasound diagnostic apparatus 10 is an apparatus that scans a subject with an ultrasound beam to generate an ultrasound image based on a reception signal obtained by the scanning. In particular, the ultrasound diagnostic apparatus 10 can also form, based on the reception signal, an ultrasound tomographic image (B-mode image) in which an amplitude intensity of a reflected wave from a scanning surface is transformed into brightness, and a Doppler image formed based on a difference (Doppler shift) in frequency between a transmitted wave and a frequency of the received wave. It should be noted that, in the present specification, the Doppler image is a concept including a color Doppler image in which a movement velocity of a blood flow or a tissue of the subject is shown by color, and a power Doppler image in which an intensity of a Doppler signal is shown by color or brightness.

An ultrasound probe 12 is a device that transmits and receives ultrasound waves to and from the subject. The ultrasound probe 12 has an oscillation element array including a plurality of oscillation elements that transmit and receive the ultrasound waves to and from the subject.

A transmission/reception unit 14 transmits a transmission signal to the ultrasound probe 12 (specifically, each oscillation element of the oscillation element array) under the control of a controller 28 (described later). As a result, the ultrasound waves are transmitted from each oscillation element toward the subject.

In addition, the transmission/reception unit 14 receives a reception signal from each oscillation element that receives the reflected waves from the subject. The transmission/reception unit 14 includes an adder and a plurality of delayers corresponding to the respective oscillation elements, and phase adjustment addition processing of aligning and adding phases of the reception signals from the respective oscillation elements is performed by the adder and the plurality of delayers. As a result, a reception beam signal in which information indicating a signal intensity and a frequency of the reflected wave from the subject is arranged in a depth direction of the subject is formed.

A signal processing unit 16 executes various types of signal processing including filter processing, such as applying a bandpass filter, and detection processing on the reception beam signal from the transmission/reception unit 14.

An image formation unit 18 forms the ultrasound tomographic image (B-mode image) based on the reception beam signal subjected to the signal processing by the signal processing unit 16. In addition, the image formation unit 18 forms the Doppler image based on the Doppler signal obtained by a Doppler signal processing unit 30 described later and a gamma curve decided by an appropriate gamma curve decision unit 40 described later.

A display controller 20 performs control of displaying the ultrasound tomographic image and the Doppler image formed by the image formation unit 18, and various other types of information on a display 22. The display 22 as a display unit is, for example, a display device configured by a liquid crystal display, an organic electro luminescence (EL), or the like.

An input interface 24 is configured by, for example, a button, a track ball, a touch panel, or the like. The input interface 24 is used to input a command from a user to the ultrasound diagnostic apparatus 10.

A memory 26 includes a hard disk drive (HDD), a solid state drive (SSD), an embedded multi media card (eMMC), a read only memory (ROM), or the like. The memory 26 stores an ultrasound diagnostic program for operating each of the units of the ultrasound diagnostic apparatus 10. It should be noted that the ultrasound diagnostic program can also be stored, for example, in a computer-readable non-transitory storage medium, such as a universal serial bus (USB) memory or a CD-ROM. The ultrasound diagnostic apparatus 10 can read and execute the ultrasound diagnostic program from such a storage medium.

In addition, as shown in FIG. 1, a learning model 34, a training data candidate database (DB) 38, and a gamma curve DB 42 are stored in the memory 26. Details of these components will be described later.

The controller 28 includes at least one of a general-purpose processor (for example, a central processing unit (CPU)) or a dedicated processor (for example, a graphics processing unit (GPU), an application specific integrated circuit (ASIC), a field-programmable gate array (FPGA), a programmable logic device, and the like). The controller 28 may be configured by the cooperation of a plurality of processing apparatuses that are present at physically separated positions, instead of being configured by one processing apparatus. The controller 28 controls each of the units of the ultrasound diagnostic apparatus 10 in accordance with the ultrasound diagnostic program stored in the memory 26.

The Doppler signal processing unit 30 forms the Doppler signal indicating the Doppler shift (difference between a transmission frequency of the ultrasound wave transmitted to the subject and a frequency of the reception beam signal) by known Doppler processing. In addition, the Doppler signal processing unit 30 calculates power of the Doppler signal by integrating the Doppler signal in a frequency direction. Further, the Doppler signal processing unit 30 performs wall filtering (low-cut filtering) to reduce a low-frequency signal component such as a body movement of the subject included in the Doppler signal. In the present embodiment, the Doppler signal processing unit 30 calculates the power of the Doppler signal, and a texture parameter specifying unit 32, which will be described later, or the appropriate gamma curve decision unit 40 performs processing based on a power distribution of the Doppler signal.

The texture parameter specifying unit 32 specifies a texture parameter appropriate for the power distribution information based on the power distribution information indicating the power distribution of the Doppler signal.

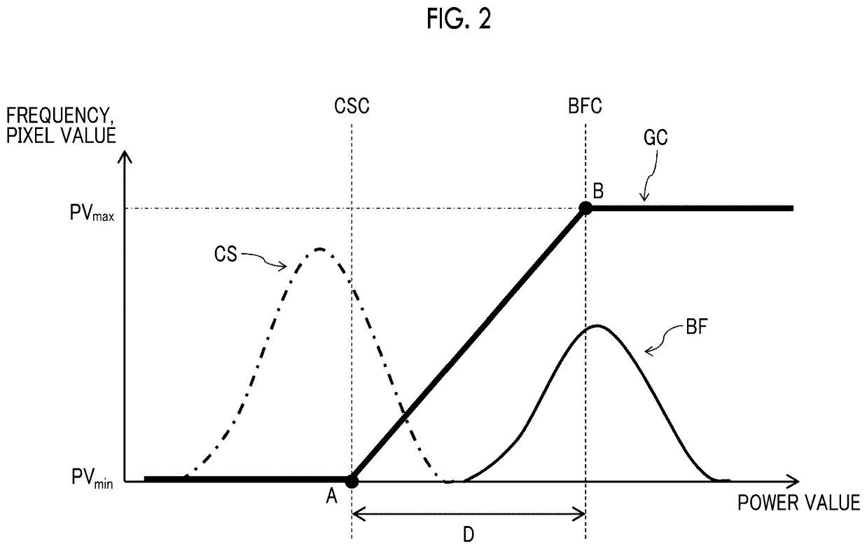

The power distribution of the Doppler signal may be, for example, a graph of a clutter signal CS and a blood flow signal BF in a two-dimensional space in which a horizontal axis represents a power value and a vertical axis represents a frequency, as shown in FIG. 2. In addition, the power distribution of the Doppler signal may be any information as long as the power distribution is information indicating the frequency with respect to the power value. For example, the power distribution of the Doppler signal may be blood flow power distribution information in which the Doppler signal is subjected to the filter processing to suppress a clutter component. The power distribution of the Doppler signal may be the power Doppler image itself.

The texture parameter is a parameter indicating a feature of a gamma curve GC (see FIG. 2). As described above, the gamma curve GC is information indicating a relationship between the Doppler signal (in the present embodiment, the power value) and the pixel value in the Doppler image (in the present embodiment, the power Doppler image). In the present embodiment, the texture parameter includes a parameter indicating a ratio of the clutter signal CS (the integrated value of the clutter signal CS in a dynamic range D in FIG. 2) included in the dynamic range D defined by the gamma curve GC to the blood flow signal BF (the integrated value of the entire blood flow signal BF in FIG. 2), and a parameter (in other words, a blood flow signal cutoff value BFC) indicating a range of the blood flow signal BF to be included in the dynamic range D.

The texture parameter specifying unit 32 specifies the texture parameter by using the learning model 34. Here, the learning model 34 and a method of training the learning model 34 via a training processing unit 36 will be described.

The learning model 34 is configured by, for example, a convolutional neural network (CNN). The learning model 34 is not limited to the CNN, and may be any model as long as the following functions are exhibited. The learning model 34 is trained using, as training data, a combination including the power distribution information and the texture parameter indicating the gamma curve appropriate for the power distribution information, to predict the texture parameter appropriate for the power distribution information and output the predicted texture parameter in a case in which the power distribution information is input.

The learning model 34 may be trained by an apparatus other than the ultrasound diagnostic apparatus 10, and the trained learning model 34 may be stored in the memory 26. However, it is considered that the appropriate (in other words, preferred) gamma curve is different for each facility (for example, a hospital or the like) in which the ultrasound diagnostic apparatus 10 is installed. Therefore, the ultrasound diagnostic apparatus 10 according to the present embodiment includes the training processing unit 36, and the training processing unit 36 executes processing of training the learning model 34. As a result, it is possible to output the texture parameter appropriate for the ultrasound diagnostic apparatus 10 by using the trained learning model 34 different for each ultrasound diagnostic apparatus 10. In addition, in order to reduce the load of the training processing or the amount of the training data in the training in each ultrasound diagnostic apparatus 10, the learning model 34 may be pre-trained by an apparatus other than the ultrasound diagnostic apparatus 10, and the training processing unit 36 may perform fine-tuning of retraining the learning model 34.

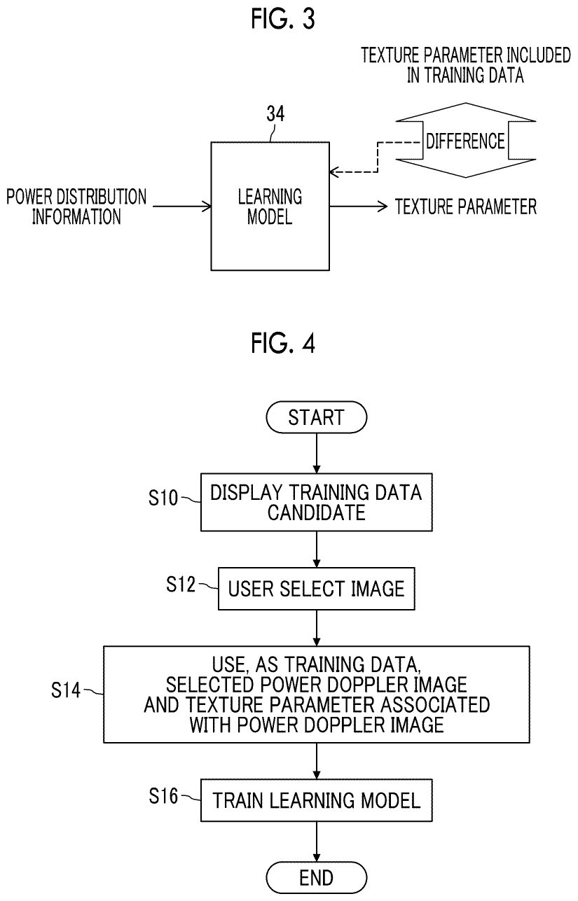

The training data used for training the learning model 34 is the combination including the power distribution information and the texture parameter indicating the gamma curve appropriate (for example, manually set by the user in the past) for the power distribution information. As shown in FIG. 3, the training processing unit 36 inputs the power distribution information included in the training data to the learning model 34. The learning model 34 predicts the texture parameter appropriate for the input power distribution information and outputs the predicted texture parameter. Based on a difference between the texture parameter output by the learning model 34 and the texture parameter included in the training data, the training processing unit 36 adjusts the parameters of the learning model 34 such that the difference is reduced. Such processing is repeated, whereby the learning model 34 is trained. The learning model 34 that has been sufficiently trained can output the texture parameter appropriate for the power distribution information with high accuracy based on the input power distribution information.

As described above, by training the learning model 34 via the training processing unit 36 of the ultrasound diagnostic apparatus 10, the trained learning model 34 can output the texture parameter appropriate for the ultrasound diagnostic apparatus 10 (in other words, a certain facility). However, in a case in which a plurality of users (for example, a doctor, a technician, and the like) use the same ultrasound diagnostic apparatus 10, there is also a case in which the texture parameter appropriate for each user is different (in other words, the preference for the gamma curve is different for each user). Therefore, the learning model 34 may be trained to predict the texture parameter appropriate for the user who uses the ultrasound diagnostic apparatus 10 and output the predicted texture parameter.

Specifically, the training processing unit 36 uses, as the training data, the combination including the power distribution information and the texture parameter indicating the gamma curve set by the user of the ultrasound diagnostic apparatus 10. Further, the training data may include user information (for example, a user ID or the like) indicating the user. In this case, the training processing unit 36 inputs the power distribution information and the user information to the learning model 34. The learning model 34 predicts the texture parameter appropriate for the user indicated by the input power distribution information and the input user information, and outputs the predicted texture parameter. Based on a difference between the texture parameter output by the learning model 34 and the texture parameter indicating the gamma curve set by the user included in the training data, the training processing unit 36 adjusts the parameters of the learning model 34 such that the difference is reduced. The learning model 34 trained in this way can output the texture parameter appropriate for the user indicated by the power distribution information and the user information with high accuracy based on the input power distribution information and user information.

Alternatively, a different learning model 34 may be prepared for each user. In this case, the memory 26 stores a plurality of learning models 34 corresponding to the plurality of users. The training processing unit 36 uses the combination including the power distribution information and the texture parameter indicating the gamma curve set by the user of the ultrasound diagnostic apparatus 10 as the training data for training the learning model 34 corresponding to the user.

It is considered that it is difficult to obtain a sufficient amount of training data for training the learning model 34 via the training processing unit 36, or it takes a considerable amount of time to obtain a sufficient amount of training data. Therefore, a plurality of combinations including the power distribution information and the texture parameter (hereinafter, referred to as “training data candidates”), which can be used as the training data, are prepared in advance to allow the user to select desired training data from among a plurality of training data candidates. Hereinafter, a flow of the processing of training the learning model 34 using the training data selected from among the training data candidates will be described with reference to the flowchart shown in FIG. 4.

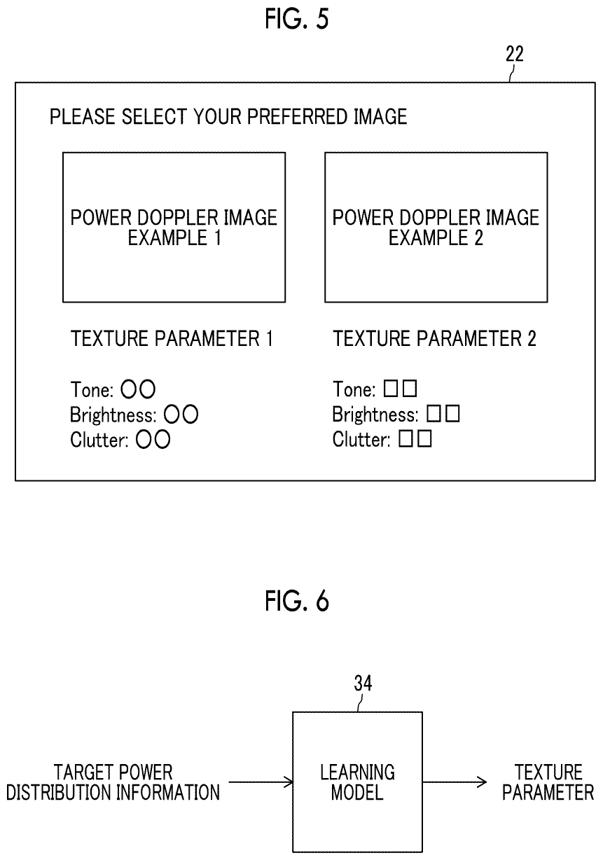

In the present embodiment, the plurality of training data candidates are stored in the training data candidate DB 38 in advance. In step S10, the plurality of training data candidates are presented to the user. In the present embodiment, the display controller 20 displays, as shown in FIG. 5, a selection screen of the training data, on which the plurality of training data candidates are displayed, on the display 22 in response to an instruction from the user. In the example of FIG. 5, the power Doppler image is displayed as the power distribution information of the training data candidates, and the texture parameter appropriate for the power distribution information is displayed below (in association with) the power Doppler image. In the example of FIG. 5, two training data candidates are displayed, but of course, the display controller 20 may display two or more training data candidates on the display 22. In a case in which the plurality of training data candidates cannot be displayed on one screen, the display controller 20 can display the plurality of training data candidates by switching the screen or the like according to a user operation.

In step S12, the user selects the desired training data from the selection screen of the training data. In this case, the training data selected by the user can be regarded as the training data including the texture parameter appropriate for the user.

In step S14, the training processing unit 36 uses, as the training data, a combination including the power Doppler image as the power distribution information selected by the user on the selection screen of the training data and the texture parameter associated with the power Doppler image.

In step S16, the training processing unit 36 trains the learning model 34 using the training data specified in step S14. It should be noted that, in a case in which the learning model 34 is provided for each user, processing of associating the learning model 34 with the user is performed after step S16.

The flowchart shown in FIG. 4 may be repeatedly executed. As a result, the learning model 34 that has been sufficiently trained can output the texture parameter appropriate for the user indicated by the power distribution information and the user information with high accuracy based on the input power distribution information and user information.

It should be noted that, in the examples of FIGS. 4 and 5, the power Doppler image is included in the training data candidate as the power distribution information, but the power distribution information included in the training data candidates may be the graph of the clutter signal CS and the blood flow signal BF shown in FIG. 2 or the blood flow power distribution information described above.

As shown in FIG. 6, the texture parameter specifying unit 32 inputs the power distribution information to be processed to the learning model 34 that has been sufficiently trained by the training processing described above. In the present specification, the power distribution information to be processed will be referred to as target power distribution information. The target power distribution information may be the graph of the clutter signal CS and the blood flow signal BF shown in FIG. 2, the blood flow power distribution information described above, or the power Doppler image. The trained learning model 34 outputs the texture parameter appropriate for the target power distribution information. The texture parameter specifying unit 32 specifies the texture parameter appropriate for the target power distribution information based on the output of the learning model 34.

As described above, in a case in which the learning model 34 is trained using the training data including the power distribution information, the user information, and the texture parameter, the texture parameter specifying unit 32 may input the target power distribution information and the user information indicating the user who is currently using the ultrasound diagnostic apparatus 10 to the trained learning model 34. It should be noted that the user information can be acquired by the controller 28 authenticating the user (for example, by input of the user ID and a password). As a result, the texture parameter specifying unit 32 can specify the texture parameter appropriate for the user, based on the output of the learning model 34.

In a case in which the learning model 34 that is different for each user is prepared, the texture parameter specifying unit 32 specifies the learning model 34 to which the target power distribution information is input, based on the user information indicating the user who is currently using the ultrasound diagnostic apparatus 10. Then, the texture parameter specifying unit 32 specifies the texture parameter appropriate for the user, based on the output of the learning model 34 by inputting the target power distribution information to the specified learning model 34.

The appropriate gamma curve decision unit 40 decides the gamma curve appropriate for the target power distribution information, based on the texture parameter appropriate for the target power distribution information, the texture parameter being specified by the texture parameter specifying unit 32. In the present specification, the gamma curve appropriate for the target power distribution information will be referred to as an appropriate gamma curve. As described above, since the texture parameter is a parameter indicating the feature of the gamma curve, the appropriate gamma curve decision unit 40 can decide the appropriate gamma curve based on the texture parameter appropriate for the target power distribution information.

As described above, in the present embodiment, since the texture parameter includes the parameter indicating the ratio of the clutter signal CS (the integrated value of the clutter signal CS in the dynamic range D in FIG. 2) included in the dynamic range D defined by the gamma curve GC to the blood flow signal BF (the integrated value of the entire blood flow signal BF in FIG. 2), and the parameter (in other words, the blood flow signal cutoff value BFC) indicating the range of the blood flow signal BF to be included in the dynamic range D, the method of deciding the appropriate gamma curve from the texture parameter will be described.

Referring to FIG. 2, the gamma curve GC is decided by the blood flow signal cutoff value BFC and a clutter signal cutoff value CSC. Therefore, the appropriate gamma curve decision unit 40 decides the appropriate gamma curve by deciding the blood flow signal cutoff value BFC and the clutter signal cutoff value CSC based on the texture parameter.

First, as for the blood flow signal cutoff value BFC, since the blood flow signal cutoff value BFC is included in the texture parameter, the appropriate gamma curve decision unit 40 can directly decide the blood flow signal cutoff value BFC from the texture parameter.

In addition, the texture parameter includes a ratio of the clutter signal CS included in the dynamic range D to the entire blood flow signal BF in the target power distribution information. The clutter signal CS included in the dynamic range D is a portion having the power value larger than the clutter signal cutoff value CSC in the entire clutter signal CS. Therefore, the appropriate gamma curve decision unit 40 decides the clutter signal cutoff value CSC such that the ratio of the portion having the power value larger than the clutter signal cutoff value CSC in the entire clutter signal CS in the entire blood flow signal BF in the target power distribution information is a ratio indicated by the texture parameter.

The appropriate gamma curve decision unit 40 decides the appropriate gamma curve such that the pixel value for the power value smaller than the clutter signal cutoff value CSC is a minimum pixel value PVₘᵢₙ (for example, a brightness value 0), the pixel value for the power value larger than the blood flow signal cutoff value BFC is a maximum pixel value PVₘₐₓ (for example, a highest brightness), and the pixel value for the power value between the clutter signal cutoff value CSC and the blood flow signal cutoff value BFC, in other words, the power value included in the dynamic range D is the pixel value corresponding to the power value. In the present embodiment, the appropriate gamma curve in the dynamic range D is a straight line connecting a point A (power value, pixel value)=(clutter suppression cutoff value, PVₘᵢₙ) and a point B (power value, pixel value)=(blood flow signal cutoff value, PVₘₐₓ).

As described above, in a case in which the learning model 34 is trained to predict the texture parameter appropriate for the user and output the predicted texture parameter, the appropriate gamma curve decision unit 40 can decide the appropriate gamma curve that is appropriate for the user, based on the texture parameter specified by the texture parameter specifying unit 32.

In the memory 26, the gamma curve DB 42 in which a plurality of types of the gamma curves are registered in advance may be stored. In such an ultrasound diagnostic apparatus 10, only the gamma curves registered in the gamma curve DB 42 may be settable. In this case, there is a case in which the appropriate gamma curve decided by the appropriate gamma curve decision unit 40 is not registered in the gamma curve DB 42. Therefore, in this case, the appropriate gamma curve decision unit 40 may select the appropriate gamma curve based on the gamma curve calculated based on the texture parameter specified by the texture parameter specifying unit 32 from among the plurality of types of the gamma curves stored in the gamma curve DB 42. For example, the appropriate gamma curve decision unit 40 may decide, as the appropriate gamma curve, the gamma curve most similar to the gamma curve obtained by the appropriate gamma curve decision unit 40 based on the texture parameter among the plurality of types of the gamma curves registered in the gamma curve DB 42. The degree of similarity between the gamma curves can be calculated, for example, according to a difference in the clutter signal cutoff value CSC and a difference in the blood flow signal cutoff value BFC between the two gamma curves.

The image formation unit 18 as a Doppler image formation unit forms the power Doppler image based on the target power distribution information and the appropriate gamma curve decided by the appropriate gamma curve decision unit 40. The power Doppler image formed in this way can be an image in which the blood flow signal BF is appropriately expressed in gradations and the influence of the clutter signal CS is reduced. The appropriate gamma curve decision unit 40 may notify the user of the decided appropriate gamma curve. For notifying the user of the appropriate gamma curve, a graph of the gamma curve GC as shown in FIG. 2 may be presented to the user, or the Doppler image formed based on the decided appropriate gamma curve may be presented to the user.

The appropriate gamma curve decision unit 40 may notify the user of the decided appropriate gamma curve. For example, the appropriate gamma curve decision unit 40 presents the graph of the gamma curve GC as shown in FIG. 2 in a pop-up form to the user. In particular, the appropriate gamma curve decision unit 40 may notify the user of the decided appropriate gamma curve in a case in which the previously decided gamma curve and the decided appropriate gamma curve are different from each other in the same scene (for example, in a case in which the same cross section is displayed or in a case in which the same examination content is used). In this case, the image formation unit 18 may wait for the instruction of the user who is notified of the appropriate gamma curve, to form the Doppler image.

In the above-described embodiment, the processing in a case in which the power Doppler image is formed as the Doppler image has been described, but the present invention can also be applied to a case in which the color Doppler image is formed as the Doppler image.

In this case, the gamma curve indicates a relationship between the velocity indicated by the Doppler signal and the pixel value in the color Doppler image. The learning model 34 is trained using, as the training data, a combination including velocity distribution information indicating a velocity distribution indicated by the Doppler signal (which may be referred to as a frequency distribution of the Doppler signal) and the texture parameter indicating the gamma curve, to predict the texture parameter appropriate for the velocity distribution information and output the predicted texture parameter in a case in which the velocity distribution information is input.

The texture parameter specifying unit 32 specifies the texture parameter appropriate for target velocity distribution information by inputting the target velocity distribution information, which is the velocity distribution information to be processed, to the trained learning model 34.

The appropriate gamma curve decision unit 40 decides the appropriate gamma curve that is appropriate for the target velocity distribution information based on the texture parameter specified by the texture parameter specifying unit 32. The same method as described above can be adopted as the method of deciding the appropriate gamma curve in this case.

Then, the image formation unit 18 as the Doppler image formation unit forms the color Doppler image in which the velocity of the blood flow or the tissue of the subject is shown, based on the target velocity distribution information and the decided appropriate gamma curve.

In addition, in the above-described embodiment, the pixel values of the entire Doppler image are made appropriate based on the appropriate gamma curve that is appropriate for the power distribution information indicating the power distribution of the entire Doppler signal or the velocity distribution information indicating the velocity distribution of the entire Doppler signal, but the pixel value of a portion of the Doppler image may be made appropriate.

In this case, the image formation unit 18 forms the Doppler image before the gamma curve adjustment, and the display controller 20 displays the Doppler image on the display 22. The user designates a portion to be optimized in the Doppler image, for example, by using a cursor. This portion will be referred to as an optimization portion. Then, the texture parameter specifying unit 32 inputs only a portion corresponding to the optimization portion of the Doppler signal to the learning model 34, and specifies the texture parameter based on the output of the learning model 34. The appropriate gamma curve decision unit 40 decides a partial appropriate gamma curve, which is the appropriate gamma curve for the optimization portion, based on the specified texture parameter. The image formation unit 18 forms the Doppler image using the decided partial appropriate gamma curve for the optimization portion. For a portion other than the optimization portion, a predetermined (for example, user-designated) gamma curve may be used, or an appropriate gamma curve decided based on the power distribution information or the velocity distribution information related to the entire Doppler signal may be used as in the above-described embodiment.

In addition, the ultrasound diagnostic apparatus 10 may automatically decide the optimization portion without depending on the user instruction. For example, a blood vessel position may be automatically detected by analyzing the ultrasound tomographic image (B-mode image) formed by the image formation unit 18 using a known technology, and a predetermined region including the blood vessel position may be automatically set as the optimization portion.

The configuration of the ultrasound diagnostic apparatus 10 according to the present embodiment is as described above. It should be noted that the transmission/reception unit 14, the signal processing unit 16, the image formation unit 18, the display controller 20, the Doppler signal processing unit 30, the texture parameter specifying unit 32, the training processing unit 36, and the appropriate gamma curve decision unit 40 provided in the ultrasound diagnostic apparatus 10 are configured by a processor. The processor includes at least one of a general-purpose processing apparatus (for example, a central processing unit (CPU)) or a dedicated processing apparatus (for example, a graphics processing unit (GPU), an application specific integrated circuit (ASIC), a field-programmable gate array (FPGA), a programmable logic device, and the like). The processor may be configured by the cooperation of a plurality of processing apparatuses that are present at physically separated positions, instead of being configured by one processing apparatus. In addition, each of the above-described units may be realized by a cooperation between hardware, such as the processor, and software.

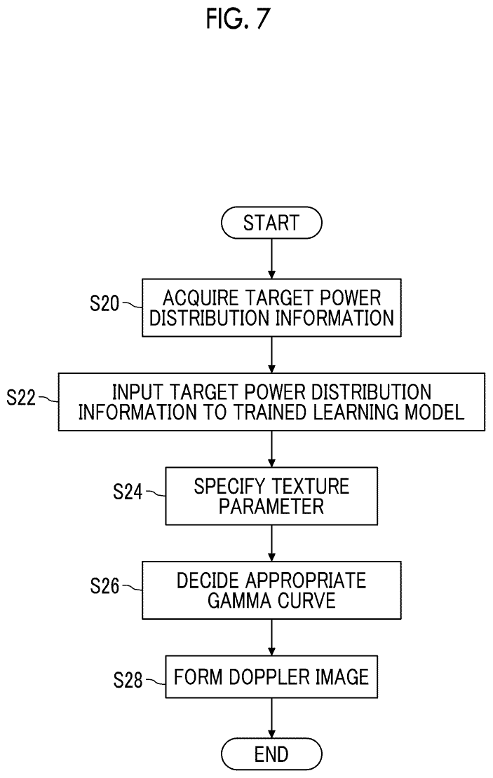

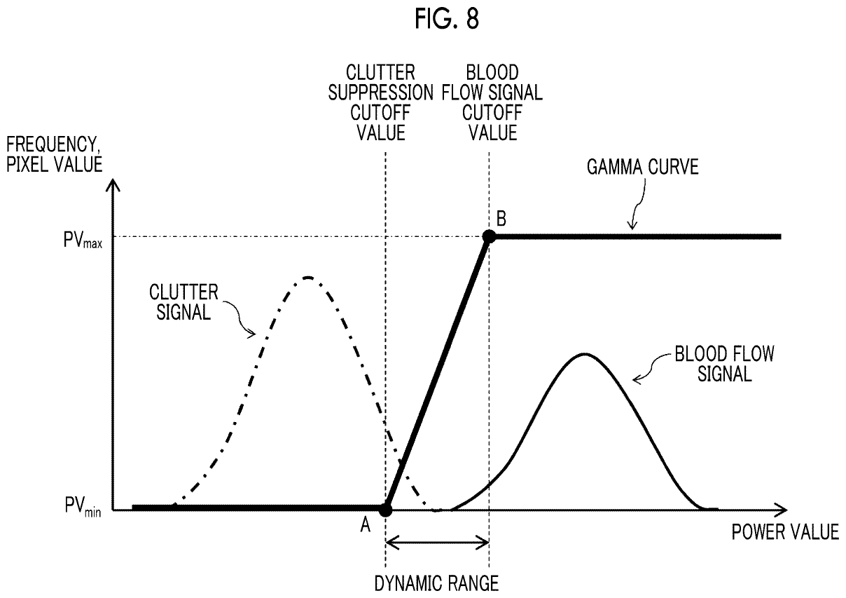

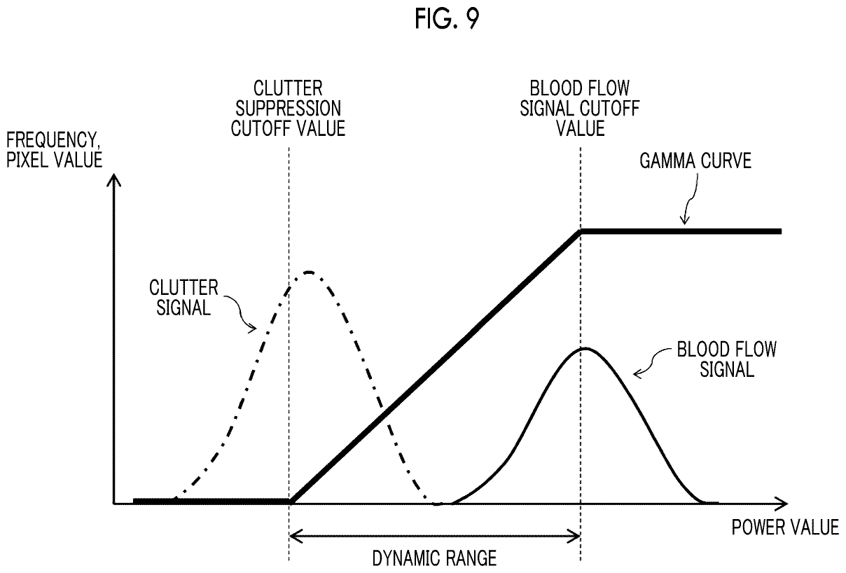

Hereinafter, a flow of the decision processing of the appropriate gamma curve will be described with reference to the flowchart shown in FIG. 7. At a start point of the flowchart shown in FIG. 7, it is assumed that the learning model 34 has been sufficiently trained.

In step S20, the Doppler signal processing unit 30 executes the Doppler processing on the reception beam data from the transmission/reception unit 14 to form the Doppler signal to be processed. Here, the Doppler signal processing unit 30 calculates the power of the Doppler signal. As a result, the target power distribution information is acquired.

In step S22, the texture parameter specifying unit 32 inputs the target power distribution information acquired in step S10 to the learning model 34.

In step S24, the learning model 34 predicts the texture parameter appropriate for the input target power distribution information, and outputs the predicted texture parameter. The texture parameter specifying unit 32 specifies the texture parameter appropriate for the target power distribution information based on the output of the learning model 34.

In step S26, the appropriate gamma curve decision unit 40 decides the appropriate gamma curve that is appropriate for the target power distribution information based on the texture parameter specified in step S24.

In step S28, the image formation unit 18 forms the power Doppler image based on the target power distribution information and the appropriate gamma curve decided in step S26.

Although the embodiment according to the present invention has been described above, the present invention is not limited to the embodiment described above, and various modifications can be made without departing from the gist of the present invention.

For example, in the above-described embodiment, although the ultrasound diagnostic apparatus 10 is the ultrasound image processing apparatus, the ultrasound image processing apparatus is not limited to the ultrasound diagnostic apparatus 10. For example, the ultrasound image processing apparatus may be a personal computer or the like. In this case, the ultrasound image processing apparatus has the functions of the image formation unit 18, the display controller 20, the display 22, the input interface 24, the memory 26, the controller 28, the Doppler signal processing unit 30, the texture parameter specifying unit 32, the training processing unit 36, and the appropriate gamma curve decision unit 40. In the ultrasound image processing apparatus, the respective units can execute the processing by receiving the reception beam data from the ultrasound diagnostic apparatus.

What is claimed is:

1. An ultrasound image processing apparatus comprising:
a texture parameter specifying unit that inputs target power distribution information to be processed to a learning model trained using, as training data, a combination including power distribution information indicating a power distribution of a Doppler signal obtained by transmitting and receiving an ultrasound wave to and from a subject, and a texture parameter indicating a gamma curve, which indicates a relationship between power of the Doppler signal and a pixel value in a Doppler image and is appropriate for the power distribution information, to predict the texture parameter appropriate for the power distribution information and output the predicted texture parameter in a case in which the power distribution information is input, and that specifies the texture parameter appropriate for the target power distribution information;
an appropriate gamma curve decision unit that decides an appropriate gamma curve that is the gamma curve appropriate for the target power distribution information, based on the specified texture parameter; and
a Doppler image formation unit that forms a Doppler image in which the power of the Doppler signal is shown, based on the target power distribution information and the appropriate gamma curve.

2. The ultrasound image processing apparatus according to claim 1, further comprising:
a training processing unit that uses, as the training data, a combination including the power distribution information and the texture parameter indicating the gamma curve set by a user, to train the learning model to predict the texture parameter appropriate for the user and output the predicted texture parameter in a case in which the power distribution information is input,
wherein the texture parameter specifying unit inputs the target power distribution information to the learning model, to specify the texture parameter appropriate for the user, and
the appropriate gamma curve decision unit decides the appropriate gamma curve that is appropriate for the user, based on the specified texture parameter.

3. The ultrasound image processing apparatus according to claim 2,
wherein the training processing unit presents a plurality of the combinations including the power distribution information and the texture parameter, which are stored in advance in a memory, to the user, and uses, as the training data, a combination including the power distribution information and the texture parameter, which is selected by the user, to train the learning model.

4. The ultrasound image processing apparatus according to claim 1,
wherein the appropriate gamma curve decision unit selects the appropriate gamma curve based on the gamma curve calculated based on the texture parameter specified by the texture parameter specifying unit from among a plurality of types of the gamma curves stored in advance in a memory.

5. The ultrasound image processing apparatus according to claim 1,
wherein the appropriate gamma curve decision unit notifies a user of the decided appropriate gamma curve.

6. An ultrasound image processing apparatus comprising:
a texture parameter specifying unit that inputs target velocity distribution information to be processed to a learning model trained using, as training data, a combination including velocity distribution information indicating a velocity distribution indicated by a Doppler signal obtained by transmitting and receiving an ultrasound wave to and from a subject, and a texture parameter indicating a gamma curve, which indicates a relationship between a velocity indicated by the Doppler signal and a pixel value in a Doppler image, to predict the texture parameter appropriate for the velocity distribution information and output the predicted texture parameter in a case in which the velocity distribution information is input, and that specifies the texture parameter appropriate for the target velocity distribution information;
an appropriate gamma curve decision unit that decides an appropriate gamma curve that is the gamma curve appropriate for the target velocity distribution information, based on the specified texture parameter; and
a Doppler image formation unit that forms a Doppler image in which a velocity of a blood flow or a tissue of the subject is shown, based on the target velocity distribution information and the appropriate gamma curve.
